SEQUENCE LISTING (10 sequences)

SEQ ID NO: 1 - protein, 69 aa, Escherichia coli
TPDCVTGKVEYTKYNDDDTFTVKVGDKELFTNRWNLQSLLLSAQITGMTVTIKTNACHNGGGFSEVIFR

SEQ ID NO: 2 - protein, 69 aa, Escherichia coli
APDCVTGKVEYTKYNDDDTFTVKVGDKELFTNRWNLQSLLLSAQITGMTVTIKTNACHNGGGFSEVIFR

SEQ ID NO: 3 - protein, 69 aa, Escherichia coli
APDCVTGKVEYTKYNDDDTFTVKVADKELFTNRWNLQSLLLSAQITGMTVTIKTTACHNGGGFSEVIFR

SEQ ID NO: 4 - protein, 70 aa, Escherichia coli
ADCAKGKIEFSKYNEDDTFTVKVDGKEYWTSRWNLQPLLQSAQLTGMTVTIKSSTCESGSGFAEVQFNND

SEQ ID NO: 5 - protein, 68 aa, Escherichia coli
ADCAKGKIEFSKYNENDTFTVKVAGKEYWTNRWNLQPLLQSAQLTGMTVTIKSNTCASGSGFAEVQFN

SEQ ID NO: 6 - protein, 70 aa, Escherichia coli
ADCAKGKIEFSKYNENDTFTVKVAGKEYWTSRWNLQPLLQSAQLTGMTVTIKSSTCESGSGFAEVQFNND

SEQ ID NO: 7 - protein, 70 aa, Escherichia coli
ADCAKGKIEFSKYNENDTFTVKVAGKEYWTSRWNLQPLLQSAQLTGMTVTIKSSTCASGSGFAEVQFNND

SEQ ID NO: 8 - protein, 68 aa, Escherichia coli
ADCAKGKIEFSKYNEDNTFTVKVSGREYWTNRWNLQPLLQSAQLTGMTVTIISNTCSSGSGFAQVKFN

SEQ ID NO: 9 - protein, 68 aa, Escherichia coli
ADCAVGKIEFSKYNEDDTFTVKVSGREYWTNRWNLQPLLQSAQLTGMTVTIISNTCSSGSGFAQVKFN

SEQ ID NO: 10 - protein, 70 aa, Escherichia coli
ADCAKGKIEFSKYNGDNTFTVKVDGKEYWTNRWNLQPLLQSAQLTGMTVTIKSNTCESGSGFAEVQFNND

(19) United States
(12) Patent Application Publication
(10) Pub. No.: US 2021/0139567 A1
(43) Pub. Date: May 13, 2021

(54) METHOD FOR DETECTING VEROTOXIN

(71) Applicants: SHIMADZU CORPORATION, Kyoto (JP); OSAKA INSTITUTE OF PUBLIC HEALTH, Osaka (JP)

(72) Inventors: Koichi KOJIMA, Kyoto (JP); Kanae TERAMOTO, Kyoto (JP); Shinichi IWAMOTO, Kyoto (JP); Yuki WAKABAYASHI, Osaka (JP); Junko SAKATA, Osaka (JP)

(73) Assignees: SHIMADZU CORPORATION, Kyoto (JP); OSAKA INSTITUTE OF PUBLIC HEALTH, Osaka (JP)

(21) Appl. No.: 17/096,203

(22) Filed: Nov. 12, 2020

(30) Foreign Application Priority Data
Nov. 13, 2019 (JP) 2019-205604

(51) Int. Cl. C07K 16/12; G01N 33/68; G01N 30/72

(52) U.S. Cl. CPC C07K 16/1232; C07K 16/2896; G01N 30/72; G01N 33/6848

(57) ABSTRACT

A method for detecting verotoxin includes: providing a sample and a molecule that binds to verotoxin; performing an operation for purifying verotoxin in the sample by using binding of the molecule and the verotoxin; and subjecting the sample obtained by the operation to a first mass spectrometry.

### INCORPORATION BY REFERENCE

The disclosure of the following priority application is herein incorporated by reference: Japanese Patent Application No. 2019-205604 filed Nov. 13, 2019

### TECHNICAL FIELD

The present invention relates to a method for detecting verotoxin.

### BACKGROUND ART

Food poisoning caused by Enterohemorrhagic Escherichia coli EC) is caused by verotoxins produced by the EHEC. Verotoxin includes a verotoxin type 1 and a verotoxin type 2 which are distinguished from each other serologically, and each of the verotoxin type 1 and the verotoxin type 2 includes a plurality of subtypes. The detection of verotoxin is important in identifying food which is a cause of food poisoning, diagnosing, or predicting the symptom.

In NPL 1, the gene of verotoxin is amplified by Polymerase Chain Reaction (PCR) and detected. This method can determine the presence or absence of the gene of verotoxin in bacteria contained in the sample, but cannot distinguish whether or not the verotoxin is expressed in the bacteria. In NPL 2, a verotoxin type 1 and a verotoxin type 2 are detected, but subtypes thereof cannot be distinguished. In NPL 3, whether or not the sample contains verotoxin is determined based on whether or not a peak is present at around a m/z value corresponding to verotoxin in mass spectrometry. However, when there is a peak corresponding to a foreign substance at around the m/z value corresponding to verotoxin, a determination of false positive is made.

### CITATION LIST

### Non-Patent Literatures

NPL 1: Seto et al., Manuals for inspection and diagnosis of “enterohemorrhagic Escherichia coli (EHEC),” Japan, National Institute of Infectious Diseases, Sep. 25, 2019

NPL 2: “NH IMMUNOCHROMATO VT 1/2 <<Instruction Manual>>First Edition, Japan, NH Food Ltd., December 2009

NPL 3: Fagerquist C K, Zaragoza W J, Sultan 0, Woo N, Quinones B, Cooley M B,0 Mandrell RE. “Top-down proteomic identification of Shiga toxin 2 subtypes from Shiga toxin-producing Escherichia coli by matrix-assisted laser desorption ionization-tandem time of flight mass spectrometry” Applied and environmental microbiology, (the United States), American Society for Microbiology, May 2014,Volume 80, Issue 9, pp. 2928-2940

### SUMMARY OF INVENTION

### Technical Problem

It is preferred that verotoxin is detected more accurately using mass spectrometry.

### Solution to Problem

The present invention according to a 1st aspect relates to a method for detecting verotoxin, the method comprising: providing a sample and a molecule that binds to verotoxin; performing an operation for purifying verotoxin in the sample by using binding of the molecule and the verotoxin; and subjecting the sample obtained by the operation to a first mass spectrometry.

### Advantageous Effects of Invention

The present invention allows verotoxin to be detected more accurately using mass spectrometry.

### BRIEF DESCRIPTION OF DRAWINGS

FIG. 1 is a flowchart illustrating flow of a method for detecting verotoxin according to one embodiment.

FIG. 2 is a table showing amino acid sequences of the respective subtypes of B subunit of verotoxin (Stx1a: SEQ ID NO: 1, Stx1c: SEQ ID NO: 2, Stx1d: SEQ ID NO: 3, Stx2a: SEQ ID NO: 4, Stx2b: SEQ ID NO: 5, Stx2c: SEQ ID NO: 6, Stx2d: SEQ ID NO: 7, Stx2e: SEQ ID NO: 8, Stx2f: SEQ ID NO: 9, and Stx2g: SEQ ID NO: 10).

FIG. 3 is a mass spectrum of a sample obtained by a purification operation using an anti-verotoxin 2 antibody in Example 1.

FIG. 4 is a mass spectrum of a sample obtained by a purification operation using an anti-verotoxin 1 antibody in Example 1.

FIG. 5 is a mass spectrum of a sample obtained by a purification operation using an anti-verotoxin 2 antibody in Example 2.

FIG. 6 is a mass spectrum of a sample obtained by a purification operation using an anti-verotoxin 1 antibody in Example 2.

### DESCRIPTION OF EMBODIMENTS

The embodiments of the present invention will be described below with reference to the drawings.

Embodiment

In a method for detecting verotoxin according to this embodiment, an operation of purifying verotoxin in a sample by using a molecule that binds to verotoxin is performed, and mass spectrometry is thereafter performed.

FIG. 1 is a flowchart illustrating steps of the method for detecting verotoxin according to this embodiment. In step S1001, a sample and a molecule that binds to verotoxin are provided.

Sample

The sample is not particularly limited as long as being a liquid, a solid or the like which contains or may contain verotoxin. For example, the sample includes a liquid, a solid or the like which contains or may contain enterohemorrhagic Escherichia coli. The sample which may contain verotoxin includes, for example, food or drink taken by or contacted with an organism such as a human having a symptom of food poisoning, and a vomit of the organism. Further, bacterial cultures or bacterial colonies obtained by culturing a bacterium in these samples, and bacterial lysates obtained from the bacterial cultures or bacterial colonies may also be appropriate samples for the method according to this embodiment. The application of the method according to this embodiment to the sample which may contain verotoxin to detect verotoxin or identify a type or a subtype of the verotoxin allows specifying food or drink which is a cause of food poisoning or allows diagnosis of or prediction of symptoms of food poisoning. The method according to this embodiment may be applied to a sample from which verotoxin has been detected. For example, the method according to this embodiment allows identification of an unknown type or subtype of the detected verotoxin, allows the use thereof for research of verotoxin, or allows confirmation of a detection result of verotoxin obtained by another method.

When whether or not verotoxin is produced is determined for one kind of microorganism in the case in which the sample contains a plurality of kinds of microorganisms, the one kind of microorganism is extracted preferably by culturing the microorganisms on a plate medium or the like, and collecting a colony obtained by the culturing. In this case, as mentioned above, a bacterial culture containing the colonies, or a bacterial lysate obtained from the colonies may be used as the sample.

Molecule that Binds to Verotoxin

Hereinafter, a molecule that binds to verotoxin is referred to as a verotoxin-binding molecule. The verotoxin-binding molecule is not limited to particular molecules as long as the sample containing verotoxin can be purified by using binding between the verotoxin-binding molecule and verotoxin. In this case, the purification of a sample refers to a relative reduction in concentration of at least some of molecules other than verotoxin in the sample compared with the concentration of verotoxin.

The verotoxin-binding molecule is preferably an antibody or globotriaosylceramide which is a receptor of verotoxin in cells. In the following embodiment, the “antibody” includes, in addition to immunoglobulin such as IgG, an immunoglobulin-like molecule which is not immunoglobulin, but has a variable region which has antigen specificity in the immunoglobulin.

A molecule bindable to a type or a subtype of verotoxin to be detected can be used as a verotoxin-binding molecule. Verotoxin is composed of one A subunit and five B subunits. A subunit is composed of an A1 subunit and an A2 subunit. The verotoxin-binding molecule may be a molecule that binds to the A1 subunit, a molecule that binds to an A2 subunit, or a molecule that binds to a B subunit.

FIG. 2 is a table showing types and subtypes of verotoxin and amino acid sequences of the respective types and subtypes of B subunit of verotoxin. The verotoxin type 1 includes subtypes of Stx1a, Stx1c, and Stx1d. An Stx1a B subunit has an amino acid sequence “TPDCVTGKVEYTKYNDDDTFTVKVGDKELFTNRWNLQSLLLSAQITGMTVTIKT NACHNGGGFSEVIFR” (SEQ ID NO: 1). An Stx1c B subunit has an amino acid sequence

“APDCVTGKVEYTKYNDDDTFTVKVGDKELFTNRWNLQSLLLSAQITGMTVTIKT NACHNGGGFSEVIFR” (SEQ ID NO: 2). An Stx1d B subunit has an amino acid sequence “APDCVTGKVEYTKYNDDDTFTVKVADKELFTNRWNLQSLLLSAQITGMTVTIKT TACHNGGGFSEVIFR” (SEQ ID NO: 3).

The verotoxin type 2 includes subtypes of Stx2a, Stx2b, Stx2c, Stx2d, Stx2e, Stx2f, and Stx2g. An Stx2a B subunit has an amino acid sequence “ADCAKGKIEFSKYNEDDTFTVKVDGKEYWTSRWNLQPLLQSAQLTGMTVTIKS STCESGSGFAEVQFNND” (SEQ ID NO: 4). An Stx2b B subunit has an amino acid sequence “ADCAKGKIEFSKYNENDTFTVKVAGKEYWTNRWNLQPLLQSAQLTGMTVTIKS NTCASGSGFAEVQFN” (SEQ ID NO: 5). An Stx2c B subunit has an amino acid sequence “ADCAKGKIEFSKYNENDTFTVKVAGKEYWTSRWNLQPLLQSAQLTGMTVTIKS STCESGSGFAEVQFNND” (SEQ ID NO: 6). An Stx2d B subunit has an amino acid sequence “ADCAKGKIEFSKYNENDTFTVKVAGKEYWTSRWNLQPLLQSAQLTGMTVTIKS STCASGSGFAEVQFNND” (SEQ ID NO: 7). An Stx2e B subunit has an amino acid sequence

“ADCAKGKIEFSKYNEDNTFTVKVSGREYWTNRWNLQPLLQSAQLTGMTVTIISN TCSSGSGFAQVKFN” (SEQ ID NO: 8). An Stx2f B subunit has an amino acid sequence “ADCAVGKIEFSKYNEDDTFTVKVSGREYWTNRWNLQPLLQSAQLTGMTVTIISN TCSSGSGFAQVKFN” (SEQ ID NO: 9). An Stx2g B subunit has an amino acid sequence “ADCAKGKIEFSKYNGDNTFTVKVDGKEYWTNRWNLQPLLQ SAQLTGMTVTIKS NTCESGSGFAEVQFNND” (SEQ ID NO: 10).

Hereinafter, a property of binding to a specific type or subtype of verotoxin and not binding to other types or subtypes of verotoxin is referred to as “selectively binding.” The verotoxin-binding molecule may be an antibody which selectively binds to verotoxin type 1, an antibody which selectively binds to verotoxin type 2, or an antibody which is bindable to the verotoxin types 1 and 2. The verotoxin-binding molecule may be an antibody which is bindable to at least one selected from the group consisting of Stx1a, Stx1c, Stx1d, Stx2a, Stx2b, Stx2c, Stx2d, Stx2e, Stx2f, and Stx2g. The verotoxin-binding molecule is desirably one whose epitope is a site which is common in each type and each subtype of the verotoxin, but is not particularly limited thereto.

The verotoxin-binding molecule may be either a monoclonal antibody or a polyclonal antibody. In the present embodiment, a plurality of kinds of molecules may be used as the verotoxin-binding molecules. The verotoxin-binding molecules may contain a plurality of kinds of antibodies having different antigen specificity or structures. In this case, the plurality of kinds of antibodies are brought into contact with the sample in the later-described purification operation.

Returning to FIG. 1, step S1003 is started after the step S1001. In the step S1003, an operation for purifying verotoxin is performed. Hereinafter, this operation is referred to as a purification operation. When the sample contains verotoxin, the verotoxin is purified by the purification operation.

The way of purifying verotoxin by the purification operation is not limited to particular ways as long as the purification is performed by using the binding of the verotoxin-binding molecule and verotoxin. In the purification operation, a reaction for binding the verotoxin-binding molecule to verotoxin is performed. For example, the sample is brought into contact with a solution containing a verotoxin-binding molecule. The purification operation of purifying verotoxin is performed preferably by separating, by an ultrafiltration, a complex obtained by binding of a verotoxin-binding molecule and verotoxin. Hereinafter, a complex of a verotoxin-binding molecule and verotoxin is also merely referred to as a complex. The purification operation of purifying verotoxin can be performed by affinity purification using a verotoxin-binding molecule. Examples of the affinity purification include immunoprecipitation, pull-down assay, and affinity chromatography using a column, a pipette chip, a microchannel, a spin column or the like. The verotoxin-binding molecule may bind to verotoxin with the verotoxin separated or unseparated into subunits. Before binding the verotoxin-binding molecule to verotoxin, an operation for separating verotoxin into subunits may be performed.

The ultrafiltration allows polymers such as proteins and fine particles dissolved in water to be separated with pores in a porous ultrafiltration membrane. Due to the variability of the pore size and the difficulty of measurement thereof, the molecular weight cutoff, rather than the pore size, is used as an indicator of the separation performance of the membrane. Each manufacturer of the ultrafiltration membranes uses different criteria to define the nominal molecular weight limit (NMWL). Molecules of a molecular weight almost equal to the molecular weight cutoff may or may not permeate the ultrafiltration membrane. In the following, the molecular weight cutoff is defined as the molecular weight with 90% rejection in a fraction curve obtained by introducing a plurality of standard substances with different molecular weights into the ultrafiltration membrane.

In the method using ultrafiltration, a verotoxin-binding molecule is brought into contact with verotoxin for binding, and the obtained complex is then separated by ultrafiltration. The A1 subunit of verotoxin has a molecular weight of about 28 kDa. The A2 subunit of verotoxin has a molecular weight of about 4 kDa. Each of the B subunits of verotoxin has a molecular weight of about 7500 to about 8000 Da. IgG of an antibody has a molecular weight of about 150 kDa. Accordingly, the complex has a molecular weight of about 150 to about 230 kDa. Therefore, the molecular weight cutoff in the ultrafiltration of the purification operation is preferably 10 kDa to 200 kDa, more preferably 40 kDa to 150 kDa, yet more preferably 70 kDa to 120 kDa. This allows the separation of foreign substances each with a mass close to the molecular weights of the A subunit and B subunits of verotoxin in a filtrate while keeping the complex on the ultrafiltration membrane. As a result, when a component kept on the ultrafiltration membrane is subjected to mass spectrometry, peaks corresponding to the respective foreign substances can be reduced at values around m/z of ions derived from the A subunit and B subunits of verotoxin. Hereinafter, m/z is used as a mass-to-charge ratio, but is not particularly limited as long as it can represent the ratio between the mass and the electric charge of an ion.

In the method using ultrafiltration, it is preferred to perform a reaction so that a free verotoxin-binding molecule binds to free verotoxin in a solution. This allows a short reaction period, and rapid purification of verotoxin compared with the case in which verotoxin binds to an antibody immobilized on a carrier. The rapid purification of verotoxin achieves detection of verotoxin in a short time, thereby particularly preferable clinically. Further, prior to the operation of binding verotoxin and the verotoxin-binding molecule, an operation of removing molecules each with a molecular weight equal to or greater than that of the antibody or molecules each with a mass greater than the molecular weight of verotoxin may be performed by microfiltration or ultrafiltration.

In the purification operation, an operation of separating verotoxin from the verotoxin-binding molecule and an operation of separating verotoxin into each subunit may be performed after separation of the complex. These operations may be performed by adding an organic solvent to the complex or adding an acid such as sulfuric acid or trifluoroacetic acid (TFA) to the complex, for example. However, when a mass spectrometry sample for MALDI is prepared as will be described later, verotoxin is separated from the verotoxin-binding molecule such as an antibody and is also separated into each subunit, by using a matrix solution, and thus these operations are not necessarily performed.

It should be noted that in the step S1003, in addition to or as substitute for ultrafiltration, microfiltration may be performed using a microfiltration membrane with comparable properties to the molecular weights of molecules to be permeated.

After the step S1003, the step S1005 is started. In the step S1005, a mass spectrometry sample is prepared.

Preparation of Mass Spectrometry Sample

Purification of the mass spectrometry sample may be performed by any method as long as purification is made according to the kind of ionization in mass spectrometry (step S1007), which will be described later, and is not limited to particular methods.

An example of performing matrix assisted laser desorption/ionization (MALDI) in the mass spectrometry will be described below. A solution that contains verotoxin or may contain verotoxin obtained by the purification operation is provided. This purified solution is desalinized using a solid-phase extraction chip or the like, a solution containing a matrix (hereinafter referred to as a matrix solution) is added to the resultant solution, which is then added on a MALDI sample plate and dried. Thus, a crystal containing the sample and the matrix is obtained. This crystal is used as a mass spectrometry sample. A matrix solution may be added to the solution obtained by the purification operation after disposing the solution on the MALDI sample plate. The kind of the matrix is not limited to particular matrices, and any of α-cyano-4-hydroxycinnamic acid (CHCA), sinapic acid, or 2,5-dihydroxybenzoic acid (DHB) may be used. As a solvent for the matrix solution, a solvent obtained by adding 0 vol % to 3 vol % trifluoroacetic acid (TFA) to a solution containing several tens of percentages by volume of an organic solvent such as acetonitrile in water may be used.

It should be noted that the mass spectrometry sample may also be prepared by using an additive for enhancing sensitivity as appropriate.

After the step S1005, the step S1007 is started. In the step S1007, the sample obtained by the purification operation is subjected to mass spectrometry. When the sample obtained by the purification operation contains verotoxin, a fraction containing verotoxin after the purification is subjected to mass spectrometry.

How to perform mass spectrometry is not particularly limited as long as ions with m/z corresponding to respective subunits of verotoxin to be detected can be mass-separated and detected. As the mass spectrometry, any mass spectrometry such as quadrupole mass spectrometry, ion trap mass spectrometry, and time-of-flight mass spectrometry may be performed. For detection of monovalent ions, a time-of-flight mass spectrometry is preferred from the viewpoint of accurately detecting each subunit of verotoxin having a high mass of several thousands Da or higher. The present embodiment allows detection of verotoxin by a single-stage mass spectrometry. The mass spectrometry may be performed using a mass spectrometer including at least one mass analyzer such as a quadrupole mass analyzer, an ion trap mass analyzer, and a time-of-flight mass analyzer. Prior to the mass spectrometry, chromatography such as liquid chromatography may be performed.

How to perform ionization in the mass spectrometry is not particularly limited, and MALDI or Electrospray Ionization (ESI) may be performed. MALDI is preferred from the viewpoint of easily producing monovalent ions and obtaining data which can be easily analyzed. In the case of MALDI, the mass spectrometry sample prepared as mentioned above is irradiated with a laser beam to ionize the sample.

In mass spectrometry, it is preferred that m/z of the ions to be mass-separated is scanned to acquire data for obtaining a mass spectrum. The data obtained by the mass spectrometry is referred to as mass spectrometry data. The mass spectrometry data is stored in a storage medium that can be referred to from a processing device such as a computer.

After the step S1007, the step S1009 is started. In the step 1009, the mass spectrometry data is analyzed. The mass spectrometry data is analyzed by a processing device such as a computer. It is preferred that mass spectrum data corresponding to the mass spectrum is created from the mass spectrometry data. For example, in the case of the time-of-flight mass spectrometry, each time of flight corresponds to the intensity of a detection signal of ions detected at the time of flight in the mass spectrometry data. The time of flight is converted into an m/z value based on calibrated data obtained in advance, and mass spectrum data in which the intensity of detected ions corresponds to the m/z value can be obtained.

Whether ions having m/z in the allowable range, based on the accuracy of the mass spectrometry, from the m/z values of ions derived from types and subtypes of the A1 subunit, the A2 subunit, and the B subunits of verotoxin are detected or not is determined. The detection of ions having m/z in the allowable range is regarded as detection of the corresponding type or subtype of verotoxin. As the m/z value of ions derived from each type or subtype of each subunit, a value obtained by past measurement or a value calculated based on the molecular weight (see FIG. 2) of each type or subtype. For example, for MALDI, assuming the monovalent ions obtained by adding a proton to each subunit are detected, a value obtained by adding the molecular weight of each subunit to the molecular weight of the proton may be used. It can be assumed that anion(s), cation(s) other than proton(s), or the like is added. In this way, at least one of a type and a subtype of the verotoxin is identified based on a mass-to-charge ratio of the detected verotoxin. In particular, for the detection of monovalent ions, it is preferred to detect the B subunits each having a low molecular weight because of being detected more accurately.

It should be noted that a control sample obtained by the same purification operation without using a verotoxin-binding molecule which binds to verotoxin to be detected may be subjected to mass spectrometry. For example, a molecule which does not bind to verotoxin may be added to a sample as a substitute for the verotoxin-binding molecule, the resultant sample may then be subjected to ultrafiltration, and a component remaining on the ultrafiltration membrane may be subjected to mass spectrometry. The mass spectrometry in the above step S1007 is referred to as a first mass spectrometry, and the mass spectrometry of the control sample is referred to as a second mass spectrometry. Whether the sample contains verotoxin may be determined based on the comparison between the mass spectrometry data obtained in the first mass spectrometry and the mass spectrometry data obtained in the second mass spectrometry. For example, the mass spectrum obtained in the first mass spectrometry is compared with a mass spectrum obtained in the second mass spectrometry, and in the case where a peak corresponding to verotoxin is present in the former, and is absent in the latter, it can be determined that the sample contains verotoxin. Accordingly, the comparison with the control sample allows verotoxin to be detected more reliably.

Aspects

It will be understood by a person skilled in the art that the above-mentioned exemplary embodiment and variations thereof are specific examples of the following aspects.

First Item

A method for detecting verotoxin according to an aspect includes: providing a sample and a molecule that binds to verotoxin; performing an operation for purifying verotoxin in the sample by using binding of the molecule and the verotoxin; and subjecting the sample obtained by the operation to a first mass spectrometry. This allows more accurate detection of verotoxin by using mass spectrometry.

Second Item

In a method for detecting verotoxin according to another aspect, the method for detecting verotoxin according to the first Item further includes: identifying at least one of a type and a subtype of verotoxin based on a mass-to-charge ratio of the verotoxin detected in the first mass spectrometry. This allows more accurate detection of a type or a subtype of verotoxin.

Third Item

A method for detecting verotoxin according to another aspect is the method for detecting verotoxin according to the first or second Item, wherein the molecule that binds to verotoxin is at least one of an antibody and globotriaosylceramide. This allows more reliable purification of verotoxin by using specificity of an antigen-antibody reaction or a ligand-receptor reaction.

Fourth Item

A method for detecting verotoxin according to another aspect is the method for detecting verotoxin according to the third Item, wherein the antibody is a polyclonal antibody. This allows the antibody to be created rapidly and easily compared with the case of creating a monoclonal antibody.

Fifth Item

A method for detecting verotoxin according to another aspect is the method for detecting verodoxin according to the third or fourth Item, wherein the molecule that binds to verotoxin is an antibody that is bindable to at least one of verotoxin type 1 and verotoxin type 2. This allows whether or not a sample contains verotoxin type 1, verotoxin type 2, or both of them to be detected.

Sixth Item

A method for detecting verotoxin according to another aspect is the method for detecting verotoxin according to the fourth or fifth Item, wherein in the purification, a plurality of kinds of antibodies are brought into contact with the sample. This allows verotoxin to be detected more reliably.

Seventh Item

A method for detecting verotoxin according to another aspect is the method for detecting verotoxin according to any one of the first to sixth Items, wherein in the operation, a solution containing a binding molecule in which the molecule is being bound to verotoxin is subjected to at least one of ultrafiltration and microfiltration. This allows molecules each with a molecular weight around the molecular weight of each subunit of verotoxin to be removed from the sample by using the difference between the molecular weight of each subunit of verotoxin and the molecular weight of the complex, and allows mass spectrometry data to be analyzed accurately.

Eighth Item

In a method for detecting verotoxin according to another aspect, the method for detecting verotoxin according to any one of the first to seventh Items further includes: subjecting a solution obtained by the same operation as the operation without using the molecule to second mass spectrometry; and determining whether or not the sample contains verotoxin based on comparison between data obtained in the first mass spectrometry and data obtained in the second mass spectrometry. This allows whether a peak corresponding to m/z of ions derived from verotoxin is a peak corresponding to verotoxin or not to be checked, and allows verotoxin to be detected more reliably.

The present invention is not limited by the embodiment. Other aspects conceivable within the scope of the technical idea of the present invention are encompassed in the scope of the present invention.

### EXAMPLES

The following shows Examples according to this embodiment, but the present invention is not intended to be limited by the Examples.

### Example 1

In Example 1, each purification operation was performed using an anti-verotoxin 1 antibody or an anti-verotoxin 2 antibody in an unknown sample, and verotoxin was detected by mass spectrometry. The following steps 1 to 5 were performed in this order.

1. An Escherichia coli strain derived from a patient with enterohemorrhagic Escherichia coli infection was cultured on a brain heart infusion (BHI) agar medium. Colonies formed by the culturing were then floated on a polymyxin B solution for 30 minutes, which was then subjected to centrifugal separation. Then, a supernatant was used as a sample solution.

2. 150 μL each of the sample solution obtained in the item 1 was dispensed in two tubes, and 150 μL of a PBS buffer solution containing 5 mM n-octyl-β-D-glucoside was added to each of the tubes. The obtained solutions were applied to an ultrafiltration device (NMWL: 100K Da, Merck Millipore, UFC510096), and the two tubes were simultaneously subjected to centrifugal separation (14,000 G, five minutes).

3. 0.5 μg of an anti-verotoxin 1 antibody (Nacalai Tesque, Inc., 01770-74) was added to one of filtrates obtained in the centrifugal separation of the item 2, and 0.5 μg of an anti-verotoxin 2 antibody (Nacalai Tesque, Inc., 01771-64) was added to the other filtrate, which were then incubated for 30 minutes.

4. Solutions after the incubation of the item 3 were applied to another ultrafiltration device (NMWL: 100 KDa), and the two tubes were simultaneously subjected to centrifugal separation. After the centrifugal separation, 100 μL of a PBS buffer solution containing 2.5 mM n-octyl-β-D-glucoside was added to each of residual fluids in ultrafiltration filters, and whole quantities were recovered.

5. The solutions recovered in the item 4 were desalinized using a solid-phase extraction chip (Agilent Technologies, Bond Elut OMIX, A57009100). The eluates after the desalinization were added on a stainless plate dropwise, and were then subjected to mass spectrometry using a mass spectrometer (Shimadzu Corporation, MALDI-8020), thereby obtaining mass spectra.

FIG. 3 is a mass spectrum obtained by mass spectrometry in the case of the purification operation using the anti-verotoxin 2 antibody. FIG. 4 is a mass spectrum obtained by mass spectrometry in the case of the purification operation using the anti-verotoxin 1 antibody. Each mass spectrum is a graph in which the horizontal axis represents m/z of ions detected, and the vertical axis indicates the intensity of the detection signal of the ions. The same applies to FIGS. 3 to 6. Comparing FIG. 3 with FIG. 4, a peak P1 with a high intensity was observed at m/z 7692.1 only in FIG. 4, and other peaks have almost the same intensities between FIGS. 3 and 4. This peak P1 as a candidate peak derived from verotoxin was compared with the table of FIG. 2, and was determined as being derived from the Stx1a B subunit of verotoxin. That is, the sample used in Example 1 was found to contain verotoxin, and the type or subtype thereof was Stx1a.

### Example 2

In Example 2, each purification operation was performed using an anti-verotoxin 1antibody or an anti-verotoxin 2 antibody in a known sample, and verotoxin was detected by mass spectrometry. The following steps 1 to 5 were performed in this order.

1. An Escherichia coli strain determined as having a gene of Stx1c by genetic testing was cultured on a trypticase soy agar (TSA) plate medium. Colonies formed by the culturing were then suspended in a physiological saline solution, which was then subjected to bacteriolysis using an ultrasonic homogenizer. Thus, a sample solution was obtained.

2. 150 μL each of the sample solution obtained in the item 1 was dispensed in two tubes, and 150 μL of a PBS buffer solution containing 5 mM n-octyl-β-D-glucoside was added to each of the tubes. The obtained solutions were applied to an ultrafiltration device (NMWL: 100K Da, Merck Millipore, UFC510096), and the two tubes were simultaneously subjected to centrifugal separation (14,000 G, five minutes).

3. 0.5 μg of an anti-verotoxin 1antibody (Nacalai Tesque, Inc, 01770-74) was added to one of filtrates obtained in the centrifugal separation of the item 2, and 0.5 μg of an anti-verotoxin 2 antibody (Nacalai Tesque, Inc., 01771-64) was added to the other filtrate, which were then incubated for 30 minutes.

4. Solutions after the incubation of the item 3 were applied to another ultrafiltration device (NMWL: 100 KDa), and the two tubes were simultaneously subjected to centrifugal separation. After the centrifugal separation, 100 μL, of a PBS buffer solution containing 2.5 mM n-octyl-β-D-glucoside was added to each of residual fluids in ultrafiltration filters, and whole quantities were recovered.

5. The solutions recovered in the item 4 were desalinized using a solid-phase extraction chip (Agilent Technologies, Bond Elut OMIX, A57009100). The eluates after the desalinization were added on a stainless plate dropwise, and were then subjected to mass spectrometry using a mass spectrometer (Shimadzu Corporation, MALDI-8020), thereby obtaining mass spectra.

FIG. 5 is a mass spectrum obtained by mass spectrometry in the case of the purification operation using the anti-verotoxin 2 antibody. FIG. 6 is a mass spectrum obtained by mass spectrometry in the case of the purification operation using the anti-verotoxin 1 antibody. Comparing FIGS. 5 with 6, a peak P2 with a high intensity was observed at m/z 7662.4 only in FIG. 6, and other peaks have almost the same intensities between FIGS. 5 and 6. This peak P2 as a candidate peak derived from verotoxin was compared with the table of FIG. 2, and was determined as being derived from the Stx1c B subunit of verotoxin. That is, the sample used in Example 2 was found to contain Stx1c of verotoxin, and it was concluded that the same type and subtype of verotoxin as detected in the genetic testing was expressed.

1. A method for detecting verotoxin, the method comprising:
providing a sample and a molecule that binds to verotoxin;
performing an operation for purifying verotoxin in the sample by using binding of the molecule and the verotoxin; and
subjecting the sample obtained by the operation to a first mass spectrometry.

2. The method according to claim 1, further comprising:
identifying at least one of a type and a subtype of verotoxin based on a mass-to-charge ratio of the verotoxin detected in the first mass spectrometry.

3. The method according to claim 1, wherein the molecule that binds to verotoxin is at least one of an antibody and
globotriaosylceramide.

4. The method according to claim 2, wherein
the molecule that binds to verotoxin is at least one of an antibody and globotriaosylceramide.

5. The method according to claim 3, wherein
the antibody is a polyclonal antibody.

6. The method according to claim 4, wherein
the antibody is a polyclonal antibody.

7. The method according to claim 3, wherein
the molecule that binds to verotoxin is an antibody that is bindable to at least one of verotoxin type 1 and verotoxin type 2.

8. The method according to claim 4, wherein
the molecule that binds to verotoxin is an antibody that is bindable to at least one of verotoxin type 1 and verotoxin type 2.

9. The method according to claim 5, wherein
the molecule that binds to verotoxin is an antibody that is bindable to at least one of verotoxin type 1 and verotoxin type 2.

10. The method according to claim 6, wherein
the molecule that binds to verotoxin is an antibody that is bindable to at least one of verotoxin type 1 and verotoxin type 2.

11. The method according to claim 4, wherein
in the purification, a plurality of kinds of antibodies are brought into contact with the sample.

12. The method according to claim 1, wherein:
in the operation, a solution containing a binding molecule in which the molecule is being bound to verotoxin is subjected to at least one of ultrafiltration and microfiltration.

13. The method according to claim 1, further comprising:
subjecting a solution obtained by a same operation as the operation without using the molecule to second mass spectrometry; and
determining whether the sample contains verotoxin or not based on comparison between data obtained in the first mass spectrometry and data obtained in the second mass spectrometry.
